(19) United States
(12) Patent Application Publication
(10) Pub. No.: US 2012/0303132 A1
(43) Pub. Date: Nov. 29, 2012

(54) STENT FOR BILE DUCT

(75) Inventors: Sang-Ho Kim, Gyeonggi-do (KR); Il-gyun Shin, Gyeonggi-do (KR); Han-Ki Kim, Gyeonggi-do (KR); Do-Hyun Park, Seoul (KR); Hwa-Sub Song, Seoul (KR); Jong-Chae Park, Gyeonggi-do (KR)

(73) Assignee: M.I.TECH CO., INC., Pyeongtaek Si, Gyeonggi-Do (KR)

(21) Appl. No.: 13/513,566

(22) PCT Filed: Dec. 3, 2009
(86) PCT No.: PCT/KR2009/007187

§ 371 Date: Aug. 10, 2012

(51) Int. Cl. A61F 2/90

(52) U.S. Cl. 623/23.7

(57) ABSTRACT

The present invention relates to a stent which is inserted into a stenosed bile duct to enlarge the bile duct. The stent includes a plurality of hooks which extend outwards from the main body of the stent at a predetermined angle and are spaced apart from each other. Therefore, when the stent is inserted into the bile duct, the hooks that extend outwards from the main body at a predetermined angle are hooked to the inner surface of the bile duct, thus preventing the stent from slipping relative to the bile duct.

### BACKGROUND OF THE INVENTION

1. Field of the Invention

The present invention relates to a stent which is inserted into a stenosed bile duct to enlarge the bile duct.

2. Background

Generally, if stenosis is caused in the lumen of the gullet, the duodenum, etc. of a human body by cancerous tissues or the like, food or the like cannot smoothly pass through the lumen. Recently, lumen enlarging devices which are called stents have been widely used. Such a stent is inserted into the lumen, thus enlarging a portion of the lumen that has been narrowed by stenosis so that food or the like can smoothly flow the lumen.

An inserting device such as a catheter is typically used to insert the stent into the stenosed portion of the lumen. The stent is configured such that the stent enlarges the stenosed portion using its own elasticity after it has been disposed in the stenosed portion.

FIG. 1 illustrates a typical stent. Referring to FIG. 1, the stent 10 is formed in a hollow cylindrical shape by weaving a metal wire 11.

The stent 10 includes a plurality of rows of metal wires, each of which extends in the circumferential direction in a zigzag manner to have peak parts and valley parts. The rows of metal wires are woven in such a way that peak parts 11a of a first row of metal wire intersect valley parts 11b of an adjacent second row of metal wire, and peak parts 11c of the second row of metal wire intersect valley parts 11d of an adjacent third row of metal wire, thus generally forming a mesh structure. The passage of the lumen that has stenosis can be enlarged by inserting the stent 10 into the stenosed portion of the lumen.

As shown in FIG. 2, bile that has been created in a liver L is transferred into a gallbladder G through left and right hepatic ducts. The bile is concentrated in the gallbladder G and then discharged from the gallbladder G before flowing into the intestines to assist the digestion of food. If the bile duct B through which bile that has been concentrated in the gallbladder G passes is involved in stenosis, the bile cannot normally flow into the intestines. In this case, the stent 10 must be inserted into the bile duct B to enlarge the stenosed portion of the bile duct B.

However, in the conventional technique, there is a problem in that after the stent 10 has been installed in the bile duct B, the stent 10 easily slips relative to the bile duct B.

### SUMMARY OF THE INVENTION

### Technical Problem

Accordingly, the present invention has been made keeping in mind the above problems occurring in the prior art, and an object of the present invention is to provide a stent for a bile duct which is configured such that it is prevented from slipping relative to the bile duct.

### Technical Solution

In order to accomplish the above object, the present invention provides a stent for a bile duct, including: a main body formed of metal wires in a hollow cylindrical shape; and a plurality of hooks provided on the main body by extending one of the metal wires of the main body outwards from the main body at a predetermined angle. The hooks are spaced apart from each other with respect to a circumferential direction of the main body.

The angle between each of the hooks and the main body may be greater than 0° and less than 90°.

The hooks may be disposed on a circumferential outer surface of the main body at positions symmetrical to each other.

At least one or more of the hooks may be arranged in the circumferential direction of the main body, thus forming a row of hooks, wherein the row of hooks may comprise a plurality of rows of hooks that are provided in a longitudinal direction of the main body.

The stent for the bile duct may further include a hook cover covering a surface of each of the hooks made of the metal wire.

The stent for the bile duct may further include a body cover covering a surface of the main body made of the metal wires. The body cover may have a hollow cylindrical shape.

The main body may include at least one row of metal wire formed by extending a metal wire in the circumferential direction in a zigzag manner such that a peak part and a valley part are formed in the row of metal wire.

### Advantageous Effects

As described above, in a stent for a bile duct according to the present invention, one or more hooks that extend outwards from a main body at a predetermined angle are hooked to the inner surface of the bile duct, thus preventing the stent from slipping relative to the bile duct. The hooks are disposed at positions spaced apart from each other so that interference between the hooks can be minimized, thus more reliably preventing the stent from slipping relative to the bile duct.

Furthermore, the hooks that are provided on the main body are formed by a metal wire that forms the stent and has superior elasticity. Thus, although the hooks are hooked to the inner surface of the bile duct to prevent the stent from slipping relative to the bile duct, damage to the inner surface of the bile duct can be prevented.

Moreover, because the hooks are made of the metal wire that has superior elasticity, when it is necessary to remove the stent from the bile duct, the hooks can be bent so that the stent can smoothly move in a direction in which it is removed. Therefore, the operation of removing the stent from the bile duct can be comparatively easily carried out.

In addition, covers are applied to the main body and the hooks, thus preventing cancer tissues or the like of the lesion from infiltrating into the stent and causing restenosis.

Further, because the stent is provided with a plurality of hooks, a slip of the stent relative to the bile duct can be minimized.

### BRIEF DESCRIPTION OF THE DRAWINGS

FIG. 1 is a perspective view showing a conventional stent;

FIG. 2 is a view showing the bile duct portion of the human body;

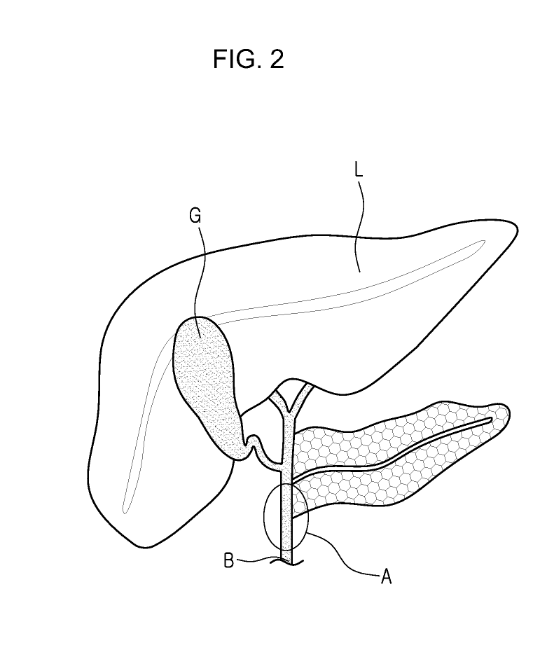

FIG. 3 is a view illustrating a stent for a bile duct, according to the present invention;

FIG. 4 is a plan view of the stent according to the present invention;

FIG. 5 is a view illustrating a portion of a main body of the stent according to the present invention;

FIG. 6 is a view showing another embodiment of a stent for a bile duct, according to the present invention;

FIG. 7 is a view showing the shape of hooks of the stent of the present invention when the stent that has been installed in portion A of FIG. 2 is removed therefrom.

<Description of the Reference Numerals in the Drawings>

100: stent for bile duct
110: main body 120: hook
130: body cover 140: hook cover
W: metal wire
PP1, PP2, PP3, PP4: first to fourth peak parts
VP1: first valley part
L: liver G: gallbladder
B: bile duct

### DETAILED DESCRIPTION OF THE INVENTION

Hereinafter, a preferred embodiment of the present invention will be described in detail with reference to the attached drawings. In the following description, detailed descriptions of well-known techniques will be omitted or summarized to make the explanation clear.

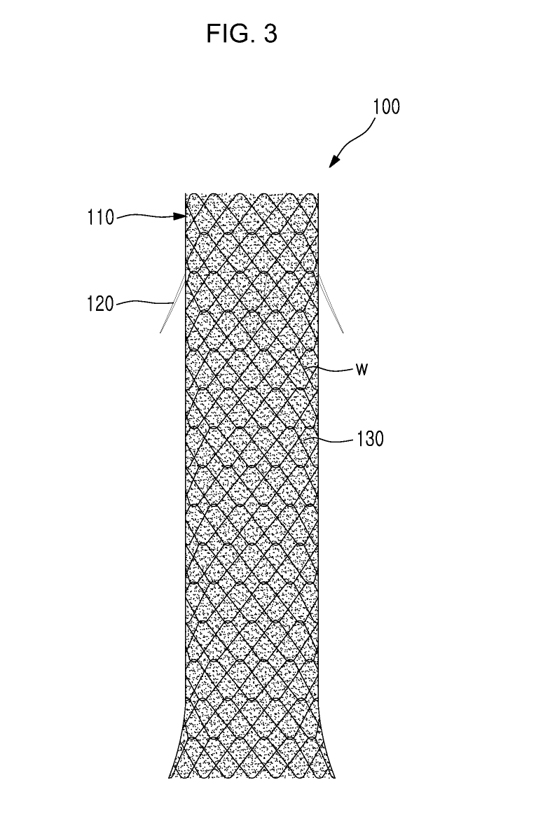

A stent 100 for a bile duct according to the present invention includes a main body 110, hooks 120, a body cover 130, and hook covers. These will be explained in detail with reference to FIGS. 3 through 6.

The main body 110 has a hollow cylindrical shape and is formed by weaving metal wires W. It is preferable for the material of the metal wires w to be a shape-memory alloy, such as a nickel-titanium alloy, having elasticity.

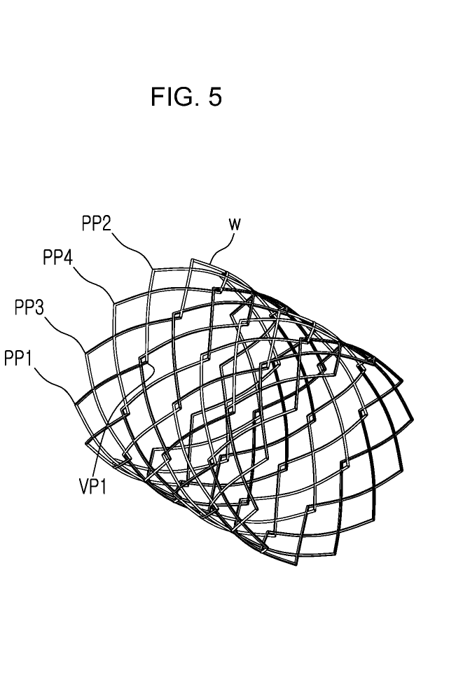

As shown in FIG. 3, the shape of the main body 110 is that of a hollow cylinder, and the main body 110 is formed by weaving the metal wires W. This will be explained in more detail with reference to FIG. 5 that shows a portion of the main body 110. Each of the metal wires W that constitute the main body 110 extend in a circumferential direction in a zigzag manner such that peak parts PP1 through PP4 and valley parts VP1 are formed, thus forming a single row. In an embodiment of the present invention, the metal wire w is woven to form the main body 110 in such a way that a peak part PP1 and a peak part PP2 of the metal wire W are formed and two peak parts PP3 and PP4 are formed between the two peak parts PP1 and PP2.

That is, in a single row of metal wire of the main body 110, because the metal wire w extends such that the first peak part PP1, the first valley part VP1, and the second peak part PP2 are successively formed and the third and fourth peak parts PP3 and PP4 are formed between the first and second peak parts PP1 and PP2, thus forming a single row, it is necessary to wind the metal wire W three times so as to form each row of metal wire.

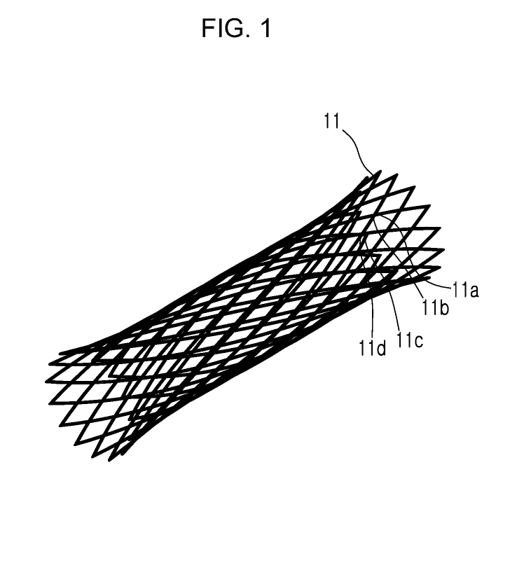

Furthermore, both ends of the main body may be extended in diameter, although this is not illustrated in this embodiment. In other words, the stent 100 of the present invention of FIG. 3 may be configured such that the diameters of both ends thereof are extended in the same manner as that of the conventional stent of FIG. 1.

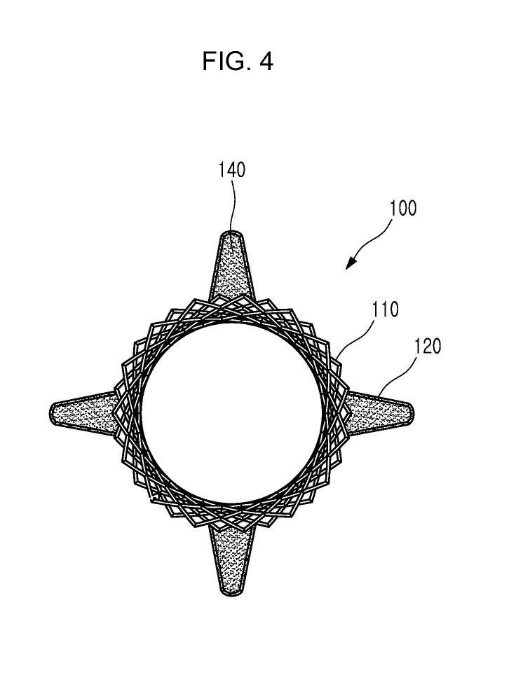

Each hook 120 is formed by extending a portion of the metal wire w outwards from the main body 110 at an inclined angle. Preferably, the hook 120 is inclined downwards, that is, in a direction in which secretion that passes through the bile duct B flows into the intestines. Here, it is preferable that a plurality of hooks 120 be formed by portions of one of the metal wires W constituting the main body 110. As shown in FIG. 4, in the stent 100 according to the present invention, the hooks 120 are provided around the main body 110 at positions spaced apart from each other in a circumferential direction.

Because the hooks 120 are spaced apart from each other in the circumferential direction of the main body 110, the possibility of interference between the hooks 120 that may be caused by the elasticity of the hooks 120 can be minimized after the stent 100 has been inserted into the bile duct B. Thereby, the effect of the hooks 120 that can prevent the stent 100 from slipping relative to the bile duct B can be maximized.

Furthermore, each hook 120 extends in the direction in which it overlaps the main body 110, and is oriented at a predetermined angle with respect to the main body 110. In the embodiment of the present invention, the angle between the hook 120 and the main body 110 is greater than 0° and less than 90°.

The reason why it is important that the hook 120 is angled to the main body 110 is because the ends of the hooks 120 of the stent 100 that has been inserted into the bile duct B can push the inner surface of the bile duct B so that the friction between the hooks 120 and the inner surface of the bile duct B can prevent the stent 100 from slipping along with secretion relative to the bile duct B.

Although the hooks 120 are angled to the main body 110, as stated above, the hooks 120 do not damage the inner surface of the bile duct B. This is because the material of the metal wire W that form the hooks 120 is a shape-memory alloy that has high elasticity so that even though the hooks 120 come into contact with the inner surface of the bile duct B, the hooks 120 elastically change in shape, thus preventing them from damaging the inner surface of the bile duct B.

Furthermore, the hooks 120 are provided around the circumferential outer surface of the main body 110 that has a hollow cylindrical shape, wherein the hooks 120 are preferably disposed at positions symmetrical to each other. In other words, the hooks 120 are provided to form a symmetrical shape with respect to the center of the hollow cylindrical main body 110. As shown in FIG. 4, in the embodiment of the present invention, four hooks 120 are symmetrically provided.

As such, because the hooks 120 extend outwards from the outer surface of the main body 110 such that they are symmetrical to each other, the hooks 120 can reliably prevent the stent 100 that has been installed in the bile duct B from slipping relative to the bile duct B downwards in, that is, in the direction in which secretion flows into the intestines. Given this, it is preferable that the elasticity of the metal wire w that forms the hooks be appropriate to restore the hooks that have been changed in shape by external force to their original states. More preferably, the material of the hooks is a shape-memory alloy, such as a nickel-titanium alloy or the like, which is the same as that of the metal wire W of the main body 110.

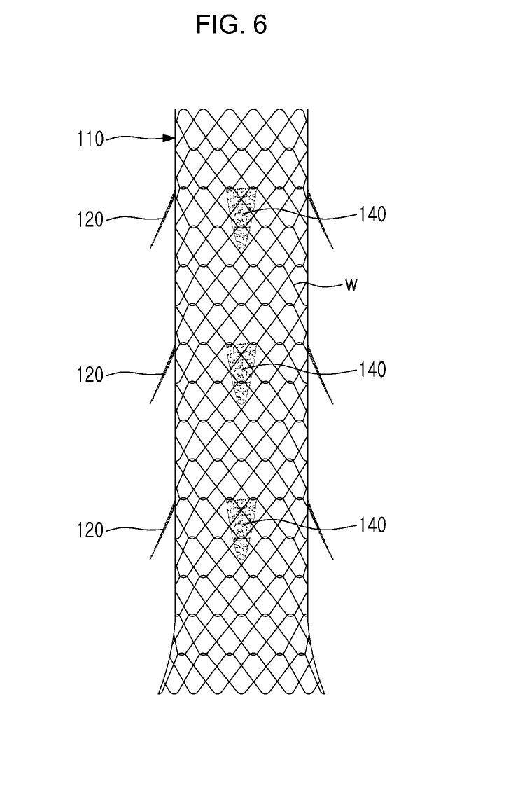

As described above, one or more hooks 120 that extend outwards from the main body 110 may be provided on one row of metal wire. Alternatively, several rows of metal wires that have hooks 120 may be provided in the longitudinal direction of the main body 110. That is, although the hooks 120 may be provided on only one row of metal wire, the stent may be configured such that the hooks 120 are arranged in the longitudinal direction of the main body 110, as shown in FIG. 6.

Referring to FIG. 6, the shape of the stent 100 of the present invention that has the hooks 120 is that of a Tannenbaum. Thanks to such a Tannenbaum shape, after the stent 100 has been inserted into the bile duct B, a slip of the stent 100 relative to the bile duct B can be minimized.

As such, the hooks 120 extend outwards from the hollow cylindrical main body 110, wherein the hooks 120 can be arranged in a variety of shapes, for example, they may be arranged in a symmetrical shape in the circumferential direction, they may be arranged in the longitudinal direction of the main body 110, they may be arranged in a spiral shape, etc.

If the stent 100 that has been installed in the bile duct B is no longer necessary, it needs to be removed. There is no likelihood of the hooks 120 being stuck to the inner surface of the bile duct B that may make it difficult to remove the stent 100 from the bile duct B.

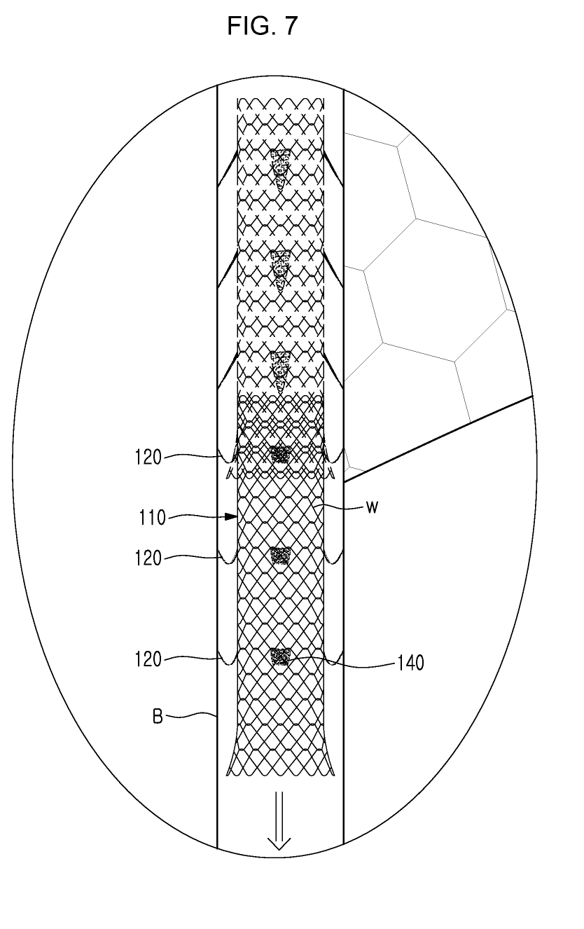

In order to remove the stent 100 from the bile duct B, it is necessary to move the stent 100 downwards, that is, in the direction in which secretion flows down in the bile duct B. When the stent 100 is moved downwards in the bile duct B by external force, as shown in FIG. 7, the hooks 120 are bent in the direction opposite to the direction in which they overlap the main body 110. In other words, the hooks 120 that have been in the state of FIG. 6 are bent in a shape in which they turn upwards.

Therefore, there is no possibility of the hooks 120 restricting the stent 100 from moving when removing the stent 100 from the bile duct B using external force. Here, the external force is incomparably stronger than the force that is applied to the stent 100 downwards by secretion that flows along the bile duct B. Only in this case can the hooks 120 bend in the direction opposite to the direction in which they overlap the main body 110.

As stated above, when external force is applied to the stent 100 to remove it from the bile duct B, the hooks 120 bend, thus making the removal of the stent 100 easy. Due to such advantages, the stent 100 of the present invention can not only be used in the bile duct B of a patient that has malignant stenosis, but can also be applied to a patient who has benign stenosis.

In the case of the patient who has malignant stenosis, the stent may have to permanently remain in the bile duct. On the other hand, in the case of the patient who has benign stenosis, for example, the bile duct B narrowing attributable to a temporary problem of the liver L, etc. so that bile cannot be normally discharged, it is only necessary to temporarily install the stent in the bile duct rather than permanently leaving the stent in the bile duct. Thus, the removal of the stent from the bile duct B is required after a predetermined time has passed. In the stent 100 for the bile duct according to the present invention, the hooks 120 easily bend when external force is applied thereto, so that the stent 100 can also be effectively used in the patient who has benign stenosis.

The shape of the body cover 130 is that of a hollow cylinder in which a passage is formed. The body cover 130 is disposed inside the main body 110 and is brought into close contact with the main body 110. The material of the body cover 130 may be polyurethane for medical use, silicon-urethane copolymer, silicone, polyamide, polyester, fluoride resin, etc.

The body cover 130 is applied to the main body 110 in such a way that it covers the whole area of the main body 110 or only a portion of the main body 110.

The hook cover 140 covers the surface of each hook 120. That is, because the hooks 120 that are formed by the metal wire w are covered with the hook covers 140, damage of the hooks 120 to the inner surface of the bile duct B can be minimized.

Although the preferred embodiment of the present invention have has been disclosed for illustrative purposes, those skilled in the art will appreciate that the present invention is not limited to the embodiment, and the scope of the present invention must be defined by the accompanying claims and their equivalents.

What is claimed is:

1. A stent for a bile duct, comprising:
a main body formed of metal wires in a hollow cylindrical shape; and
a plurality of hooks provided on the main body by extending one of the metal wires of the main body outwards from the main body at a predetermined angle,
the hooks being spaced apart from each other with respect to a circumferential direction of the main body.

2. The stent for the bile duct according to claim 1, wherein the angle between each of the hooks and the main body is greater than 0° and less than 90°.

3. The stent for the bile duct according to claim 1, wherein the hooks are disposed on a circumferential outer surface of the main body at positions symmetrical to each other.

4. The stent for the bile duct according to claim 1, wherein at least one or more of the hooks are arranged in the circumferential direction of the main body, thus forming a row of hooks,
wherein the row of hooks comprises a plurality of rows of hooks that are provided in a longitudinal direction of the main body.

5. The stent for the bile duct according to claim 1, further comprising
a hook cover covering a surface of each of the hooks made of the metal wire.

6. The stent for the bile duct according to claim 1, further comprising
a body cover covering a surface of the main body made of the metal wires, the body cover having a hollow cylindrical shape.

7. The stent for the bile duct according to claim 1, wherein the main body comprises at least one row of metal wire formed by extending a metal wire in the circumferential direction in a zigzag manner such that a peak part and a valley part are formed in the row of metal wire.
